(19) United States
(12) Patent Application Publication
(10) Pub. No.: US 2006/0241450 A1
(43) Pub. Date: Oct. 26, 2006

(54) Ultrasound guided tissue measurement system

(75) Inventors: Luiz B. Da Silva, Danville, CA (US); Charles L. Chase, Dublin, CA (US)

Correspondence Address: John P. Wooldridge, 114 Honu'ea Pl, Kihei, HI, 96753, US

(73) Assignee: BioTelligent Inc.

(21) Appl. No.: 11/408,353

(22) Filed: Apr. 21, 2006

Related U.S. Application Data
(63) Continuation-in-part of application No. 11/375,873, filed on Mar. 13, 2006.
Application No. 11/375,873 is a continuation-in-part of application No. 10/803,574, filed on Mar. 17, 2004, now abandoned.
(60) Provisional application No. 60/455,536, filed on Mar. 17, 2003.
(60) Provisional application No. 60/674,135, filed on Apr. 21, 2005.
(60) Provisional application No. 60/760,196, filed on Jan. 18, 2006.

(51) Int. Cl. A61B 8/00

(52) U.S. Cl. 600/443

(57) ABSTRACT

A tissue guidance system integrates an ultrasound imaging device with a tissue diagnostic probe. Ultrasound imaging is used to guide the tissue probe to the target area. Measurements made by the probe can then be analyzed to determine tissue type or state (e.g., malignant or benign).

This application claims priority to U.S. Provisional Patent Application Ser. No. 60/674,135, titled: “Ultrasound Guided Tissue Measurement System”, filed Apr. 21, 2005, incorporated herein by reference.

This application is a continuation-in-part of U.S. patent application Ser. No. 11/375,873, filed Mar. 13, 2006, titled “Optical Biopsy System With Single Use Needle Probe”, incorporated herein by reference. This application is a continuation-in-part of U.S. patent application Ser. No. 10/803,574 filed Mar. 17, 2004, incorporated herein by reference. This application claims priority to U.S. Provisional Patent Application Ser. No. 60/455,536, filed Mar. 17, 2003, incorporated herein by reference. This application claims priority to U.S. Provisional Patent Application Ser. No. 60/760,196, filed Jan. 18, 2006, incorporated herein by reference.

### BACKGROUND OF THE INVENTION

1. Field of the Invention

The invention relates to a tissue diagnostic system for determining tissue type or state that uses ultrasound imaging to guide placement of the measurement probe.

2. Description of Related Art

Various methods and devices have been developed to measure physical characteristics of tissue in an effort to distinguish between cancerous and non-cancerous tissue. For example, U.S. Pat. No. 5,303,026 to Strobl et al. (the Strobl patent) is directed to an apparatus and method for spectroscopic analysis of scattering media such as biological tissue. More specifically, the Strobl patent is directed to an apparatus and method for real-time generation and collection of fluorescence, reflection, scattering, and absorption information from a tissue sample to which multiple excitation wavelengths are applied.

U.S. Pat. No. 5,349,954 to Tiemann et al. is directed to an instrument for characterizing tissue. The instrument includes a hollow needle for delivering light, from a monochromator, through the needle to a desired tissue region. Mounted in the shaft of the needle is a photodiode having a light sensitive surface facing outward from the shaft for detecting back-scattered light from the tissue region.

U.S. Pat. No. 5,800,350 to Coppleson et al. is directed to an apparatus for tissue type recognition. In particular, the patent discusses apparatus intended to include a probe configured to contact the tissue and subject the tissue to a plurality of different stimuli such as electrical, light, heat, sound and magnetism and to detect plural physical responses to the stimuli. The patent discusses the use of a processor for processing the responses in combination in order to categorize the tissue. The processing is to occur in real-time with an indication of the tissue type (e.g., normal, pre-cancerous/cancerous, or unknown) intended to be provided to an operator of the apparatus.

U.S. Pat. No. 6,109,270 to Mah et al. (the Mah patent) is directed to a multimodality instrument for tissue characterization. In one configuration, a system with a multimodality instrument for tissue identification is intended to include a computer-controlled motor driven heuristic probe with a multisensory tip. U.S. patent application Ser. No. 09/947,171 to Hular et al. is directed to a probe intended to incorporate optical fibers and electrical conductors into a compact needle like probe to measure the optical and electrical properties of tissue.

All these devices are positioned or located by the physician by palpating the lesion or in some cases with x-ray or ultrasound guided imaging. Ultrasound imaging alone now provides accurate guidance but lacks the contrast to accurately determine tissue state. U.S. Pat. No. 6,171,249 to Chin et al. is directed to an ultrasound imaging device intended to include an integrated working channel for guiding core biopsies. Unfortunately, the working channel as described is impractical for a stiff multi-sensor needle probe. Therefore, there is a need for an integrated ultrasound imaging and tissue measurement device that can provide the physician with the information to guide the diagnostic procedure. The present invention addresses this need.

### SUMMARY OF THE INVENTION

It is an object of the present invention to provide a method and a system that can be used by physicians to accurately position a tissue measurement probe and provide a diagnosis.

It is another object of the invention to provide a system that can be used by surgeons to determine whether a suspicious lesion is cancer or normal tissue.

These and other objects will be apparent to those skilled in the art based on the disclosure herein.

In normal use the present invention is used by a physician to measure tissue properties along a desired path. The desired path is determined by the physician based on ultrasound imaging of the area of interest. In one embodiment the system is used to diagnose breast cancer. In this embodiment the ultrasound imaging transducer is placed in contact with the breast such that the suspicious lesion is visible in the ultrasound image. The physician then inserts the probe into the tissue and moves it within the image plane of the ultrasound transducer until it reaches the desired point within the lesion. Measurements made by the probe are then combined with information from the ultrasound image to determine whether the lesion is cancer or normal tissue.

In one embodiment of the system the tissue measurement probe is constrained by a probe guide that includes an angle encoder. The angle is measured by the control electronics and used by the image display software to show the predicted path of the probe.

In another embodiment of the system the probe guide is motorized to drive the probe into the tissue at a selected velocity. This has the advantage of sampling tissue properties in a more uniform manner.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 is an illustration showing an embodiment of the present invention.

FIG. 2 shows an application of the device used to diagnose breast cancer.

FIG. 3 is an ultrasound transducer design that integrates a probe guidance element.

FIG. 4 is an illustration of a typical image.

### DETAILED DESCRIPTION OF THE INVENTION

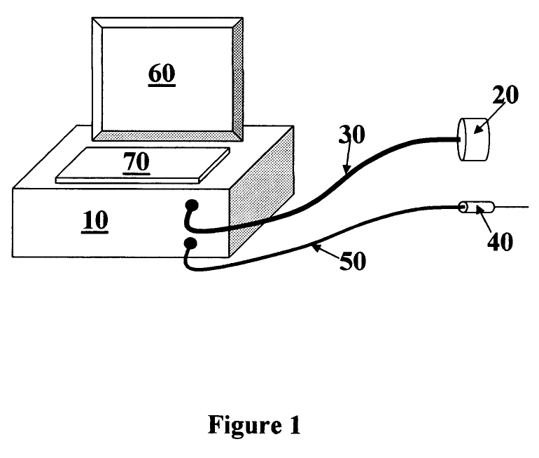

The present invention provides a system that can be used by physicians to accurately position a tissue measurement probe and provide a diagnosis. FIG. 1 shows the major components of the measurement system. A control electronics module 10 connects to an ultrasound imaging transducer 20 through a cable 30. A tissue measuring probe 40 connects to the control module 10 through cable 50. In normal use the control electronics 10 collect data from the ultrasound transducer 20 and tissue probe 40 and processes the data for display on monitor 60. A user interface 70 is used by the user to control data acquisition, data display and analysis.

The ultrasound imaging transducer 20 can be mechanically scanned or a phased array design (see, e.g., “The Physics of Medical Imaging” Ed. Steve Webb (1988), incorporated herein by reference and “Ultrasound in Medicine” Ed. F. A. Duck, A. C. Baker, H. C. Starritt (1997), incorporated herein by reference). Although a two dimensional imaging ultrasound transducer is sufficient, a three dimensional imaging ultrasound transducer could also be used. The ultrasound transducer operating frequency is selected to effectively image to the maximum tissue depth necessary for probe placement. For most applications the ultrasound frequency will be in the range of 1-10 MHz.

The tissue measuring probe 40 can be any needle like device with at least one sensor near or at the tip of the probe. The sensor must be capable of measuring tissue properties. One possible such probe is a cancer optical biopsy probe as described in, Bigio et al., “Diagnosis Of Breast Cancer Using Elastic-Scattering Spectroscopy: Preliminary Clinical Results”, Jour. Biomed. Optics 5, 221-228 (2000), incorporated herein by reference, and U.S. Pat. No. 5,303,026, incorporated herein by reference. Another possible probe is a multi sensor cancer biopsy probe of U.S. Pat. No. 6,109,270, incorporated herein by reference or U.S. patent application Ser. No. 09/947,171, incorporated herein by reference. For improved contrast, the probe 40 is coated with an echogenic layer (e.g., ECHO—COAT® produced by STS Biopolymers Inc., 336 Summit Point Drive, Henrietta, N.Y. 14467) that enhances the reflection and scatter of ultrasound.

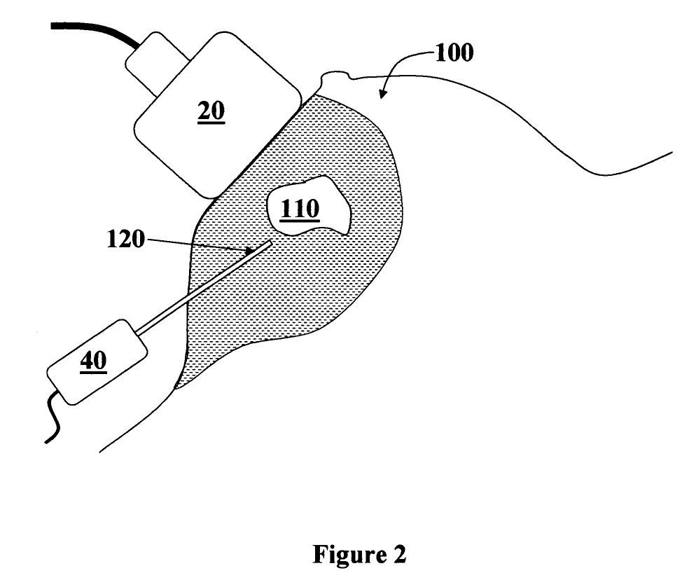

FIG. 2 shows how the device would be positioned to guide a breast cancer diagnostic procedure. The imaging ultrasound transducer 20 is positioned in contact with the breast 10 to image the lesion (or desired target area) 110 and the transducer 20 is used to track the position of the needle-like section of the tissue measuring probe 120. To improve ultrasound imaging an acoustic gel can be applied between the transducer 20 and the breast 100.

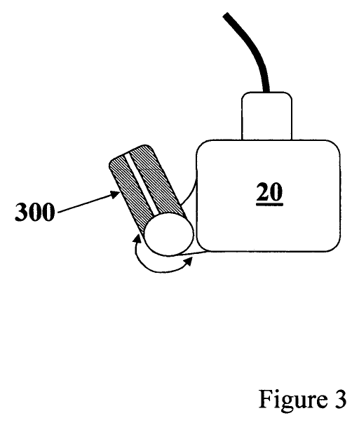

When the ultrasound transducer is a two dimensional imager, then it is important that the probe lies within the imaging plane of the transducer. The width of the image plane is controlled by the transducer design and is typically less than 5 mm. To insure that the probe is within the image plane the ultrasound transducer can incorporate a probe guide 3 as shown in FIG. 3. The probe guide can rotate within the imaging plane to allow the user to direct the probe at the lesion. The rotation hub 310 is marked so that the user can also directly set the angle of insertion. In one embodiment of the display software, lines can be overlaid on the computer image showing the probe trajectory for selected angles. The user can use this overlay map to select the probe insertion angle. In another embodiment the hub 310 contains a position encoder that is read by the control electronics to determine the current angle setting. This data is used by the software to display the predicted trajectory of the probe. In another embodiment the rotation hub 310 contains a motorized translation stage that advances the probe at a constant velocity. This insures that the probe is inserted into tissue accurately. In addition, an encoder on the motorized translation stage can be monitored to provide the software with additional information about the position of the distal tip of the probe within the tissue.

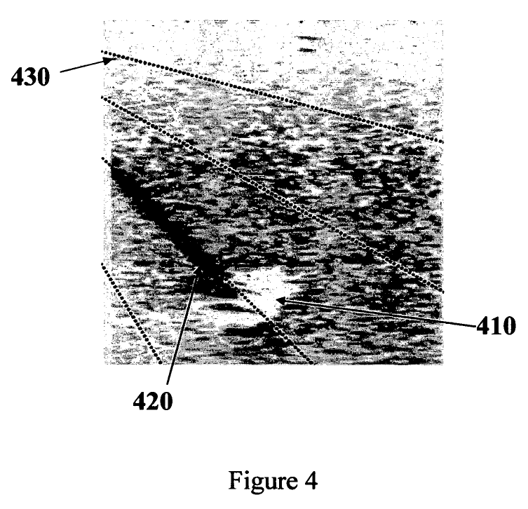

FIG. 4 shows an example of the image that would be displayed on the monitor 60 during the procedure. The image shows the lesion 410 and the instantaneous position of the probe 420. To guide the user dashed lines 430 are overlaid on the image for a few selected insertion angles (e.g., 15, 30, 45, and 60 degrees). An image acquisition rate of at least 5 Hz is desired in order to allow the user to accurately monitor probe insertion. Using the imaging information the user can verify that the probe enters the lesion 410. Information from the ultrasound image can be used along with probe sensor measurements to determine tissue type and state (e.g., cancer or normal).

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

The foregoing description of preferred embodiments of the invention is presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated.

We claim:

1. An apparatus, comprising:
a tissue measurement probe; and
an ultrasound imaging transducer; and
means for collecting and analyzing first data from said tissue measurement probe and second data from said ultrasound imaging transducer to determine the position of said tissue measurement probe within tissue.

2. The apparatus of claim 1, further comprising an ultrasound energy source attached to said ultrasound imaging transducer.

3. The apparatus of claim 2, wherein said ultrasound energy source is configured to provide ultrasound energy within a frequency range from about 1-10 MHz.

4. The apparatus of claim 1, wherein said means comprises a computer system including a computer readable medium having software that analyses said first data and said second data to determine said position of said tissue measurement probe within said tissue

5. The apparatus of claim 1, wherein said ultrasound imaging transducer comprises a phased array.

6. The apparatus of claim 1, wherein said tissue measuring probe comprises a needle like device with at least one sensor near or at the tip of the probe.

7. The apparatus of claim 6, wherein said probe comprises a cancer optical biopsy probe.

8. The apparatus of claim 1, wherein said probe comprises an echogenic layer to enhance the reflection and scatter of ultrasound.

9. The apparatus of claim 1, wherein said ultrasound transducer comprises an image plane and means for guiding said probe within said image plane.

10. The apparatus of claim 1, wherein said ultrasound transducer comprises an image plane and a probe guide for guiding said probe within said image plane.

11. The apparatus of claim 10, wherein said probe guide limits the placements said probe to areas within said image plane.

12. The apparatus of claim 11, wherein said probe guide limits the rotation of said probe to areas within said imaging plane.

13. The apparatus of claim 11, wherein said probe guide comprises a rotation hub.

14. The apparatus of claim 13, wherein said rotation hub is marked to set an angle of insertion of said probe.

15. The apparatus of claim 4, further comprising a display, wherein said software includes display software showing probe trajectory for selected angles.

16. The apparatus of claim 13, wherein said hub comprises a position encoder that is read by the control electronics to determine the current angle setting.

17. The apparatus of claim 16, further comprising a display, wherein said software includes display software showing probe trajectory for selected angles.

18. The apparatus of claim 13, wherein said hub comprises a motorized translation stage.

19. The apparatus of claim 18, wherein said stage is configured to advance said probe at a constant velocity.

20. The apparatus of claim 19, wherein said hub comprises a position encoder that is read by the control electronics to determine the current angle setting.

21. The apparatus of claim 20, further comprising a display, wherein said software includes display software showing probe trajectory for selected angles.

22. A method for identifying tissue comprising inserting a tissue measurement probe into tissue, wherein said tissue measurement probe comprises a structure as recited in any one of claims 1-21.
